(19) United States
(12) Patent Application Publication
(10) Pub. No.: US 2024/0226140 A1
(43) Pub. Date: Jul. 11, 2024

(54) NITRIC OXIDE AND MOLECULAR HYDROGEN GENERATING COMPOSITIONS

(71) Applicant: Azulent, LLC, Las Vegas, NV (US)

(72) Inventors: Eugene P. Pittz, Las Vegas, NV (US); Chen Chen, Las Vegas, NV (US)

(21) Appl. No.: 18/405,865

(22) Filed: Jan. 5, 2024

Related U.S. Application Data
(60) Provisional application No. 63/576,004, filed on Jan. 9, 2023.

(51) Int. Cl. A61K 33/00; A61K 9/00; A61K 9/06; A61K 31/194; A61K 33/06; A61K 33/30; A61K 36/21; A61K 47/36

(52) U.S. Cl. CPC A61K 33/00; A61K 9/0014; A61K 9/06; A61K 31/194; A61K 33/06; A61K 33/30; A61K 36/21; A61K 47/36

(57) ABSTRACT

Compositions and methods for generating molecular hydrogen and nitric oxide. An example composition can include a base metal powder, a nitrogen-containing salt, and an acid. When mixed with water, the composition can generate and sustain at least one of nitric oxide or molecular hydrogen. The compositions can be used in methods of treating a condition in a subject. One example method of treating a condition in a subject can include administering to the subject the composition including the base metal powder, nitrogen-containing salt, and acid.

### CROSS REFERENCE TO RELATED APPLICATIONS

This application claims priority to U.S. Provisional Patent Application No. 63/576,004, filed on Jan. 9, 2023, which is incorporated herein by reference.

### BACKGROUND

Molecular hydrogen (H₂) can have many health benefits for humans and animals. Molecular hydrogen can provide health benefits when delivered by various routes, including intravenous (IV), oral, transdermal, and inhalation. In some cases, molecular hydrogen can function as an antioxidant by direct reaction with hydroxyl radicals and peroxynitrite, while leaving signaling oxygen species, such as superoxide and hydrogen peroxide, unchanged. Some health benefits of molecular hydrogen can include anti-aging, general wellness, prevention and treatment of diseases, reducing tissue damage, slowing progression of chronic degenerative diseases, body weight control, and others. Some methods that have been used to provide molecular hydrogen include the electrolysis of water, reaction of metals and metal hydrides with water, direct splitting of water through vibrating piezoelectric zinc oxide microfibers in aqueous solution, pressurizing molecular hydrogen into water in containers resistant to permeation, and others.

Several commercial electrolysis devices are available for generating molecular hydrogen from water. Many of these devices utilize ‘purified’ water. ‘Purified’ water is defined here as water that is free of contaminants. Of most concern is the presence of those electrolytes in water that can form deposits on the electrodes of the electrolysis device and render it useless. ‘Purified’ water can be produced by distillation, reverse osmosis, ion exchange or any method that results in water that is free of, or very low in, ions and organic contaminants. For electrolysis, the appropriate pH can be near neutral and the water can be free of electrolytes that can degrade the electrodes. Use of electrolytes greatly reduces the lifetime of electrodes. However, these device often produce a very low output of molecular hydrogen. To increase the output of molecular hydrogen from ‘purified’ water, very high voltages, which may be unsafe for consumers, can be used.

Nitric oxide (NO) is another gaseous compound that can provide a variety of health benefits. Nitric oxide is a signaling molecule in both the plant and animal kingdoms. It can boost oxygen delivery to cells, relaxes and expands blood vessels, improves circulation, reduces arterial plaque, prevents blood clots, regulates blood pressure, helps erectile dysfunction, improves exercise, it is involved in neurotransmission, it prevents cognitive decline, and has antimicrobial activity. Nitric oxide can be produced by the body through at least two pathways. In the arginine-citrulline pathway, endothelial nitric oxide synthase or other nitric oxide synthase enzymes convert arginine to nitric oxide. In the nitrate to NO pathway, mouth bacteria convert nitrate to nitrite, which is then swallowed and converted to nitric oxide in the stomach and beyond.

### SUMMARY

The present disclosure describes compositions for generating molecular hydrogen and/or nitric oxide. In some examples, a composition can include a base metal powder, a nitrogen-containing salt, and an acid. When the composition is mixed with water, the composition can generate and sustain at least one of nitric oxide or molecular hydrogen. In certain examples, the base metal powder can include zinc, magnesium, calcium, or a combination thereof. The acid can be an organic acid. The nitrogen-containing salt can be a nitrate salt, a nitrite salt, or a combination thereof.

In another example, a method of treating a condition in a subject can include administering to the subject the composition including the base metal powder, the nitrogen-containing salt, and the acid. In certain examples, the composition can be administered topically. In further examples, the composition can include water and can be in the form of a hydrogel, paste, gum, or liquid.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

None.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a metal” includes reference to one or more of such substances and reference to “the acid” refers to one or more of such compounds.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, “base metal” can refer to metals that are not considered precious. For example, base metals can include metals that are not gold, silver, or platinum. In certain examples, base metals can include zinc, magnesium, and calcium.

As used herein, “average particle size” refers to a number-average of the particle size of particles in a composition. The particle size refers to the diameter of spherical particles or, for irregularly shaped particles, the longest dimension of the particle.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” and “at least one of A, B, or C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### EXAMPLE EMBODIMENTS

As explained above, molecular hydrogen gas and nitric oxide gas can both provide health benefits to humans and lower animals. However, many of the available sources of molecular hydrogen and nitric oxide do not provide a sufficient amount of these gases or a sufficiently sustained release of these gases to allow for the maximum benefit. Molecular hydrogen can be short-lived inside the body because it can easily diffuse into and out of tissues. However, some effects of hydrogen, such as reduction of tissue damage, slowing of aging, slowing of progression of chronic degenerative diseases, and body weight control, can be increased when the body is exposed to the sustained presence of hydrogen over an extended period of time. Similarly, nitric oxide produced in vivo by the body's natural processes has a short half-life of only two milliseconds in the body. Greater effects may be achieved by longer, more sustained exposure to nitric oxide.

The compositions described here can provide release of molecular hydrogen, nitric oxide, or both. Therefore, the compositions can be used to provide the various health effects that are possible with molecular hydrogen and nitric oxide. The compositions can be administered in various ways, including intravenous, injection, oral ingestion, topical application, or others. Typically, the compositions can be administered orally and topically. The compositions can also be used to immediately generate hydrogen and nitric oxide outside of a living human or animal.

In some examples of the present technology, a composition can include a base metal powder, a nitrogen-containing salt, and an acid. The composition can be a dry powder composition, which can be mixed with water to activate the composition. Once activated, the composition can immediately begin to generate hydrogen and/or nitric oxide. Without being bound to a particular mechanism, in some examples the base metal powder can react with water to form molecular hydrogen according to Equation (1):

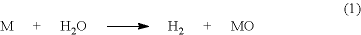

where M is the metal of the base metal powder. Again, without being bound a particular mechanism, it is believed that the metal catalyzes the generation of NO from nitrate or nitrite through Equation (2) and Equation (3):

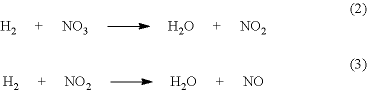

The base metal powder can be made up of particles of a non-precious metal (i.e., not gold, silver, or platinum). In some examples, the base metal can be an alkaline earth metal or a transition metal. In certain examples, the base metal can be calcium, magnesium, zinc, or a combination thereof. In further examples, the base metal can be an alkaline earth, and in some specific examples the alkaline earth metal can be calcium or magnesium. In other examples, the base metal powder can include calcium, magnesium, zinc, aluminum, copper, barium, iron, tin, manganese, or a combination thereof. In various examples, the base metal can be a metal that is reactive with water to form molecular hydrogen (H₂). In some examples, the composition can consist of, or consist essentially of, the base metal, the nitrogen-containing salt, and the acid, and optionally where the base metal is a single metal. In one example, the composition can include citric acid, zinc metal powder, and magnesium nitrate. As one very specific example, a single dose can be prepared as a composition of 2.0 grams citric acid, 0.76 grams zinc metal powder, 0.4 grams magnesium nitrate which is suitable for adding to 100 mL of water (e.g. including aqueous beverages). In some examples, the composition further includes excipients. Optional excipients can include erythritol, konjac glucomannan, and xanthan gum which form a gel. In other examples, the composition can include sweeteners. Sweeteners can be particularly useful to offset a sour taste associated with citric acid, for example. Non-limiting examples of sweeteners can include sucrose, stevia, allulose, sucralose, and the like.

The base metal powder can have an average particle size from 100 nm to 500 μm, or from 100 nm to 100 μm, or from 100 nm to 50 μm, or from 100 nm to 10 μm, or from 100 nm to 5 μm, or from 100 nm to 1 μm, or from 1 μm to 5 μm, or from 5 μm to 10 μm, or from 10 μm to 50 μm, or from 50 μm to 100 μm, or from 100 μm to 500 μm.

The compositions described herein can be prepared in a dry powder state in some examples. In a dry composition, the base metal powder can be present at a concentration from 0.005 wt % to 50 wt % with respect to the total weight of the dry composition, or from 0.5 wt % to 50 wt %, or from 5 wt % to 50 wt %, or from 20 wt % to 50 wt %, or from 30 wt % to 50 wt %, or from 1 wt % to 30 wt %, or from 1 wt % to 20 wt %, or from 10 wt % to 30 wt %, or from 20 wt % to 30 wt %. When the composition is mixed with water, to prepare the composition to be administered to a subject, the base metal powder can be present at a concentration from 0.005 wt % to 25 wt % with respect to the total weight of the wet composition, or from 0.005 wt % to 10 wt %, or from 0.005 wt % to 5 wt %, or from 0.005 wt % to 1 wt %, or from 0.005 wt % to 0.5 wt %, or from 0.005 wt % to 0.25 wt %, or from 0.005 wt % to 0.05 wt %, or from 0.05 wt % to 0.25 wt %, or from 0.25 wt % to 0.5 wt %, or from 0.5 wt % to 1 wt %, in some examples.

In addition to the base metal powder, the compositions can also include a nitrogen-containing salt. In some examples, the nitrogen-containing salt can include a nitrate salt, a nitrite salt, or a combination thereof. These salts can be made up of a nitrate or nitrite anion with a cation. Examples of cations that can be included in the nitrogen-containing salt can include sodium, potassium, magnesium, calcium, zinc, ammonium, aluminum, barium, bismuth, copper, iron, lithium, manganese, silver, and combinations thereof. In certain examples, the nitrogen-containing salt can be sodium nitrite, sodium nitrate, magnesium nitrite, magnesium nitrate, calcium nitrite, calcium nitrate, or a combination thereof. In further examples, the cation can be an ion of the same metallic element as the base metal powder.

The nitrogen-containing salt can be present in the composition, in a dry state, at a concentration from 0.01 wt % to 50 wt %, with respect to the total dry weight of the composition, in some examples. In further examples, the concentration of the nitrogen-containing salt can be from 0.1 wt % to 50 wt %, or from 1 wt % to 50 wt %, or from 5 wt % to 50 wt %, or from 10 wt % to 50 wt %, or from 20 wt % to 50 wt %, or from 30 wt % to 50 wt %, or from 5 wt % to 30 wt %, or from 5 wt % to 20 wt %. When the composition is mixed with water, the concentration of the nitrogen-containing salt in the wet state can be from 0.01 wt % to 50 wt %, or from 0.01 wt % to 30 wt %, or from 0.01 wt % to 10 wt %, or from 0.01 wt % to 5 wt %, or from 0.01 wt % to 1 wt %, or from 0.01 wt % to 0.1 wt %, or from 0.1 wt % to 1 wt %, or from 0.1 wt % to 3 wt %, or from 0.1 wt % to 5 wt %, or from 0.5 wt % to 3 wt %, or from 0.5 wt % to 5 wt %.

Some nitrogen-containing salts can be part of a natural product. The natural product can be a plant or derived from a plant. In certain examples, the natural product can be a botanical powder, a vegetable powder, a fruit powder, or a combination thereof. These natural products can be included in the compositions described herein to provide the nitrogen-containing salt. In some examples, the natural product can be present in the composition, in a dry state, at a concentration from 0.5 wt % to 80 wt % with respect to the total dry weight of the composition. In further examples, the concentration can be from 1 wt % to 80 wt %, or from 5 wt % to 80 wt %, or from 20 wt % to 80 wt %, or from 40 wt % to 80 wt %, or from 60 wt % to 80 wt %, or from 40 wt % to 60 wt %, or from 20 wt % to 40 wt %, or from 5 wt % to 20 wt %. When water is added, the concentration of the natural product can be from 0.1 wt % to 40 wt %, or from 0.1 wt % to 20 wt %, or from 0.1 wt % to 10 wt %, or from 0.1 wt % to 5 wt %, or from 0.1 wt % to 1 wt %, or from 0.5 wt % to 1 wt %, or from 0.5 wt % to 5 wt %, or from 0.5 wt % to 10 wt %, or from 0.5 wt % to 20 wt %, with respect to the total weight of the wet composition.

Examples of natural plant-based powders that can be used in the composition include botanical powders such as green coffee bean powder, danshen root powder (Radix saliva mitorrhizae), snake gourd fruit powder (Fructus trichosanthis), longstamen onion bulb powder (Bulbus allii macrostemi), sanchi powder (Radix notoginseng), ginseng powder (Radix ginseng), bing pian powder (Borneolum syntheticum), and borneol powder (Cinnamomum), and combinations thereof. Further examples can include vegetable powders such as arugula powder, beet powder, cabbage powder, celery powder, cress powder, fennel powder, leek powder, spinach powder, lettuce powder, mustard green powder, parsley powder, swiss chard powder, leafy chicory powder, kohlrabi powder, radish powder, and combinations thereof. Other examples can include fruit powders such as watermelon powder, apple powder, banana powder, grape powder, kiwi powder, nectarine powder, peach powder, pomegranate powder, pear powder, orange powder, and combinations thereof. In certain examples, a combination of one or more vegetable powders and one or more fruit powders can be used.

The compositions described herein can also include an acid. In some examples, the acid can be an organic acid. In certain examples, the acid can be citric acid, ascorbic acid, or a combination thereof. Other example organic acids can include lactic acid, folic acid, oxalic acid, acetic acid, salicylic acid, glycolic acid, mandelic acid, kojic acid, hyaluronic acid, and combinations thereof.

The acid can be present in the composition, in a dry state, at a concentration from 0.05 wt % to 70 wt % with respect to the total dry weight of the composition. In further examples, the concentration can be from 0.05 wt % to 35 wt %, or from 0.05 wt % to 20 wt %, or from 0.05 wt % to 10 wt %, or from 0.05 wt % to 5 wt %, or from 0.05 wt % to 1 wt %, or from 1 wt % to 5 wt %, or from 5 wt % to 10 wt %, or from 10 wt % to 20 wt %, or from 20 wt % to 35 wt %, or from 35 wt % to 70 wt %. When water is added to the composition, the concentration of the acid in the wet composition can be from 0.005 wt % to 35 wt %, or from 0.005 wt % to 20 wt %, or from 0.005 wt % to 10 wt %, or from 0.005 wt % to 5 wt %, or from 0.005 wt % to 3 wt %, or from 0.005 wt % to 1 wt %, or from 0.005 wt % to 0.5 wt %, or from 0.005 wt % to 0.1 wt %, or from 0.05 wt % to 0.1 wt %, or from 0.1 wt % to 0.5 wt %, or from 0.5 wt % to 1 wt %, or from 1 wt % to 3 wt %, or from 3 wt % to 5 wt %, or from 5 wt % to 10 wt %, in some examples.

The amount of acid can be sufficient to provide a pH at which hydrogen and/or nitric oxide can be generated by the composition. In particular, the composition can be mixed with a certain amount of water, and then the pH of the wet composition can be in a range that allows hydrogen and/or nitric oxide to be generated by the composition. In some examples, the pH of the wet composition can be less than 6, or less than 5, or less than 4, or less than 3. In further examples, the pH of the wet composition can be less than 4.1, or less than 3.9, or less than 3.7, or less than 3.5, or less than 3.4, or less than 3.2, or less than 2.9. In still further examples, the pH can be from 1 to 6, or from 2 to 5, or from 2 to 4.5, or from 2 to 4.1, or from 2 to 4, or from 2 to 3.9, or from 2 to 3.7, or from 2 to 3.4, or from 2 to 3.2, or from 2 to 2.9.

The composition can also include a hydrogel forming polymer. When the composition is mixed with water, the polymer can become hydrated and form a hydrogel. Examples of hydrogel forming polymers can include konjac glucomannan, xanthan gum, chitosan, chitosan oligosaccharide, kappa carrageenan, lambda carrageenan, sulfated polysaccharide, alginate, pectin, and combinations thereof.

In some examples, the hydrogel forming polymer can be present in the composition, in a dry state, at a concentration from 5 wt % to 70 wt % with respect to the total dry weight of the composition. In further examples, the concentration can be from 5 wt % to 35 wt %, or from 5 wt % to 20 wt %, or from 5 wt % to 10 wt %, or from 10 wt % to 20 wt %, or from 20 wt % to 35 wt %, or from 35 wt % to 70 wt % with respect to the total dry weight of the composition. When water is added to the composition, the concentration of the hydrogel forming polymer in the wet composition can be from 0.05 wt % to 35 wt %, or from 0.05 wt % to 20 wt %, or from 0.05 wt % to 10 wt %, or from 0.05 wt % to 5 wt %, or from 0.05 wt % to 1 wt %, or from 0.05 wt % to 0.5 wt %, or from 0.05 wt % to 0.1 wt %, or from 0.1 wt % to 0.5 wt %, or from 0.5 wt % to 1 wt %, or from 1 wt % to 5 wt %, or from 5 wt % to 10 wt %, or from 10 wt % to 20 wt %.

Depending on the ingredients that are included in the dry composition and the amount of water that is mixed with the composition, the composition in its wet state can have a variety of different consistencies. In some examples, the composition can be in the form of a hydrogel, paste, gum, or liquid. The dry composition can be mixed with water at a weight ratio from 1:1,000 to 1:1 (weight of dry ingredients:weight of water) in some examples. In further examples, the weight ratio of dry ingredients to water can be from 1:1,000 to 1:5, or from 1:1,000 to 1:10, or from 1:1,000 to 1:50, or from 1:1,000 to 1:100, or from 1:1,000 to 1:500, or from 1:500 to 1:100, or from 1:100 to 1:50, or from 1:50 to 1:10, or from 1:10 to 1:5, or from 1:5 to 1:1.

The present disclosure also describes methods of using the compositions described herein. As explained above, the compositions can be used to generate molecular hydrogen, nitric oxide, or a combination of both of these gases. The molecular hydrogen and nitric oxide can be useful for treating a variety of conditions. Therefore, in some examples the compositions can be used in methods of treating a condition in a subject. For example, a method of treating a condition in a subject can include administering to the subject a composition as described above. The subject can be a human or a non-human animal such as a non-human mammal, bird, or reptile. In certain examples, the subject can have a condition such as aging skin, diabetic ulcers, burns, bacterial infection, viral infection, fungal infection, hair loss, mouth ulcers, herpes infection, gingivitis, periodontal disease, halitosis, esophagitis, lower esophageal spasms, esophageal ulcers, gastroparesis, gastric ulcers, SIBO (small intestinal bacterial overgrowth), Helicobacter pylori infection, or a combination thereof. These conditions can be treated using molecular hydrogen, nitric oxide, or a combination thereof.

Methods of administering the composition to a subject can depend on the condition that is being treated. In some examples, the composition can be applied topically to the skin. This can be useful for treating skin conditions such as aging skin, ulcers, burns, skin infections, hair loss, and other skin conditions. In some cases, applying the compositions described herein to the skin can increase blood circulation to the area of the skin where the composition is applied. The compositions described herein can also be administered orally. In some examples, the compositions can be formulated as a mouthwash to treat conditions of the mouth, gums, teeth, throat, and so on. Some conditions that can be treated this way include mouth ulcers, herpes infection, gingivitis, periodontal disease, halitosis, esophagitis, and others. The compositions can also be swallowed orally in some examples. Some conditions that can be treated by swallowing the composition can include lower esophageal spasms, esophageal ulcers, gastroparesis, gastric ulcers, SIBO (small intestinal bacterial overgrowth), Helicobacter pylori infection, and others. In other examples, the composition can be applied intranasally to generate molecular hydrogen and/or nitric oxide that can be inhaled. This can be used to treat conditions of the nose, sinuses, airway, lungs, and others. Although dosages can vary, as a general guideline, the composition can be provided in a single dose of about 3 to 12 grams per dose. Such doses may be taken twice a day in one example. These can be provided as individually packaged doses or can be provided in a larger container with a corresponding measuring scoop, for example.

The compositions can also be used for other methods that are not methods of treatment. For example, a method of generating molecular hydrogen or nitric oxide can include mixing a base metal powder, a nitrogen-containing salt, an acid, and water in amounts sufficient to generate molecular hydrogen, nitric oxide, or a combination thereof. The method can also include applying the mixture to a location where molecular hydrogen or nitric oxide is desired.

In some examples, the composition can generate a therapeutically effective amount of nitric oxide and molecular hydrogen for an extended time period. As used herein, “sustain” as in “sustained NO” or “sustained H₂” can refer to a sustained, continuous release of these gases for a time period. In some examples, the time period over which the release of NO and/or H₂ is sustained can be from 0.1 hour to 100 hours, or from 0.1 hour to 60 hours, or from 0.1 hour to 50 hours, or from 0.1 hour to 30 hours, or from 0.1 hour to 15 hours, or from 0.1 hour to 10 hours, or from 0.1 hour to 5 hours, or from 0.1 hour to 3 hours, or from 0.1 hour to 1 hour, or from 1 hour to 3 hours, or from 3 hours to 5 hours, or from 5 hours to 15 hours, or from 15 hours to 30 hours, or from 30 hours to 50 hours, or from 50 hours to 60 hours.

In further examples, the therapeutically effective amount of nitric oxide generated by the composition can be a concentration from 50 ppb to 5,000 ppb (in parts by weight with respect to the total weight of the wet composition), and in some cases 150 ppm. In further examples, the concentration of NO in the composition can be from 50 ppb to 3,000 ppb, or from 50 ppb to 2,000 ppb, or from 50 ppb to 1,000 ppb, or from 50 ppb to 800 ppb, or from 50 ppb to 500 ppb, or from 50 ppb to 300 ppb, or from 50 ppb to 150 ppb, or from 150 ppb to 300 ppb, or from 300 ppb to 500 ppb, or from 500 ppb to 800 ppb, or from 800 ppb to 1,000 ppb, or from 1,000 ppb to 2,000 ppb, or from 1 ppm to 200 ppm, or from 25 ppm to 175 ppm.

The therapeutically effective amount of H₂ generated by the composition can be a concentration from 50 ppb to 3,000 ppb in some examples. In further examples, the concentration of H₂ in the composition can be from 50 ppb to 2,000 ppb, or from 50 ppb to 1,600 ppb, or from 50 ppb to 1,200 ppb, or from 50 ppb to 1,000 ppb, or from 50 ppb to 800 ppb, or from 50 ppb to 600 ppb, or from 50 ppb to 400 ppb, or from 50 ppb to 200 ppb, or from 50 ppb to 100 ppb, or from 100 ppb to 200 ppb, or from 200 ppb to 400 ppb, or from 400 ppb to 600 ppb, or from 600 ppb to 800 ppb, or from 800 ppb to 1,000 ppb, or from 1,000 ppb to 1,200 ppb, or from 1,200 ppb to 1,600 ppb, or from 1,600 ppb to 2,000 ppb.

### Example 1

A study was conducted to determine if molecular hydrogen (H₂)-infused water and, independently, aqueous magnesium metal powder, in the presence of citric acid and sodium nitrate can generate and sustain both H₂ and nitric oxide (NO). All ingredients and test products were obtained online from Amazon, eBay or other readily available vendors. Nitric oxide was measured with an Interscan GasD® 8000 Nitric oxide detector from Interscan (United States). H₂ was measured with a Trustlex H₂ detector from Trustlex Inc. (Japan). The pH was measured with a calibrated ExTech ExStik® II pH meter from Extech (United States). Nitrite (NO₂) was estimated using Berkeley test strips. Nitrate (NO₃) was measured with a Horiba LAQUAtwin™ NO₃ meter from Horiba (Japan).

For testing with H₂-infused water, single-dose packets containing citric acid and sodium nitrate were mixed with different H₂-infused waters and measurements taken about 5-minutes thereafter. For testing with MgMP, single-dose packets containing MgMP, citric acid and sodium nitrate were mixed with distilled water and measurements were taken at various time points afterward. Table 1A shows the compositions that were tested. Table 1B shows the time points and measured concentrations of NO, H₂, NO₂, NO₃, and the pH.

TABLE 1A

| | Citric | Sodium | Magnesium | H₂ | |
|---|---|---|---|---|---|
| Test | Acid | Nitrate | Metal Powder | concentration | |
| No. | (wt %) | (wt %) | (wt %) | in water (ppb) | Mixing Method |
| 1 | 0.7 | 0.1 | 0 | 762 | Harsh, Frother |
| 2 | 0.7 | 0.1 | 0 | 421 | Harsh, Frother |
| 3 | 0.7 | 0.1 | 0 | 921 | Harsh, Frother |
| 4 | 0 | 0 | 0 | 780 | Harsh, Frother |
| 5 | 2 | 0.1 | 0 | 760 | Gentle, Stick |
| 6 | 2 | 0.8 | 0 | 813 | Gentle, Stick |
| 7 | 1 | 0.2 | 0.2 | 0 | Harsh, Frother |
| 8 | 1 | 0.2 | 0.2 | 0 | Harsh, Frother |
| 9 | 1 | 0.2 | 0.2 | 0 | Harsh, Frother |
| 10 | 1 | 0.2 | 0.2 | 0 | Harsh, Frother |
| 11 | 1 | 0.2 | 0.2 | 0 | Harsh, Frother |

TABLE 1B

| Test | Time | | | | NO₂ | NO₃ |
|---|---|---|---|---|---|---|
| No. | (hrs) | NO (ppb) | H₂ (ppb) | pH | (micromolar) | (ppm) |
| 1 | 0.1 | 0.0 | 0 | 2.7 | 20 | 510 |
| 2 | 0.1 | 0.0 | 0 | 3.0 | 20 | 520 |
| 3 | 0.1 | 0.0 | 0 | 3.0 | 20 | 600 |
| 4 | 0.1 | ND | 0 | ND | ND | ND |
| 5 | 0.1 | 0.0 | 505 | 2.2 | 20 | 950 |
| 6 | 0.1 | 0.0 | 452 | 2.2 | 20 | 5700 |
| 7 | 0.1 | 886 | 755 | 3.25 | 870 | 1100 |
| 8 | 3 | 785 | 603 | 3.43 | 870 | 890 |
| 9 | 8 | 500 | 788 | 3.81 | 870 | 900 |
| 10 | 19 | 225 | 768 | 4.08 | 870 | 750 |
| 11 | 30 | 112 | 648 | 4.34 | 870 | 740 |

The results of tests No. 1-6 demonstrate that mixing H₂-infused water with citric acid (CA) and sodium nitrate does not result in the production of nitric oxide—even when H₂ persists in the solutions. Tests No. 7-11 demonstrate that the combination of Magnesium metal powder (MgMP), citric acid (CA) and sodium nitrate does result in the production of nitric oxide where H₂ and NO persist in the solutions—for up to 30-hours. From these results it can be concluded that H₂-infused water does not interact with sodium nitrate and an acid to generate nitric oxide. However, MgMP does interact with sodium nitrate and an acid to generate nitric oxide. The MgMP interaction with sodium nitrate and an acid to generate nitric oxide is unexpectedly long-lasting due to the residue of MgMP as observed on the bottom of the reaction cup.

### Example 2

The rate of hydrogen loss was further tested by measuring H₂ concentration in water that was infused with H₂ generated by electrolysis, but which did not contain any other reactants. The H₂ dissipated rapidly from the H₂-infused water. This was compared with a composition that included distilled water (not infused with H₂) mixed with 1 wt % citric acid, 0.2 wt % sodium nitrate, and 0.2 wt % magnesium metal powder. The time course of loss of H₂, from the reaction cup described in Example 1, is shown for the formula 0.2% MgMP, 1% citric acid, 0.2% sodium nitrate in distilled water. The results are shown in Table 2.

TABLE 2

| | Time | | | | NO₂ | NO₃ |
|---|---|---|---|---|---|---|
| Composition | (hrs) | NO (ppb) | H₂ (ppb) | pH | (micromolar) | (ppm) |
| H₂-infused | 0.1 | ND | 904 | 5.6 | ND | ND |
| water | | | | | | |
| H₂-infused | 1 | ND | 883 | 5.3 | ND | ND |
| water | | | | | | |
| H₂-infused | 3 | ND | 629 | 5.3 | ND | ND |
| water | | | | | | |
| H₂-infused | 8 | ND | 72 | 5.6 | ND | ND |
| water | | | | | | |
| H₂-infused | 18 | ND | 0 | 5.6 | ND | ND |
| water | | | | | | |
| CA, NaNO₃ | 0.1 | 886 | 755 | 3.25 | 870 | 1100 |
| and MgMP | | | | | | |
| CA, NaNO₃ | 3 | 785 | 603 | 3.43 | 870 | 890 |
| and MgMP | | | | | | |
| CA, NaNO₃ | 8 | 500 | 788 | 3.81 | 870 | 900 |
| and MgMP | | | | | | |
| CA, NaNO₃ | 19 | 225 | 768 | 4.08 | 870 | 750 |
| and MgMP | | | | | | |
| CA, NaNO₃ | 30 | 112 | 648 | 4.34 | 870 | 740 |
| and MgMP | | | | | | |
| CA, NaNO₃ | 52 | 41 | 658 | 4.59 | 870 | 790 |
| and MgMP | | | | | | |

These results show that the hydrogen in the H₂-infused water dissipated completely at a time between 8 hours and 18 hours after the hydrogen was infused into the water by electrolysis. In contrast, the composition that included CA, NaNO₃, and MgMP generated a sustained release of hydrogen even as long as 52 hours after mixing the ingredients with distilled water. The sustained release of H₂ in the reaction cup containing the formula 0.2% MgMP, 1% citric acid, 0.2% sodium citrate in distilled water can be due to one or all of the following factors. First a dark residue was left at the bottom of the container, which is presumed to be magnesium metal particles. These may continuously cause the generation of H₂. Second, the presence of citric acid and sodium nitrate together with magnesium metal powder can cause the sustained release of H₂. Third, NO may transform into NO₂ or NO₃ by reacting with water in a reaction that also releases H₂ gas, although this is less likely.

### Example 3

Compositions were prepared with an aqueous gel. The aqueous gel included 7 wt % erythritol, 0.7 wt % glucomannan and 0.7 wt % xanthan gum in water. Some compositions were made with H₂-infused water and an acid and a source of nitrate, but without any base metal particles. Other compositions were made with distilled water, an acid, a source of nitrate, and base metal particles. The compositions were prepared gravimetrically as dry powder compositions and contained in 2×2″ single-dose packets. For testing, a single-dose packet was mixed with distilled water.

The results showed that mixing nitrate containing powder that does not contain a base metal with a nitrate, even if H₂-infused water is present, that NO will not be generated. On the other hand, when a base metal is present, along with an acid (citric acid) and a source of nitrate, then NO is generated. If a base metal, a nitrate source and an acid that lowers pH to a determined level are combined, then the combination can generate NO. It appears from these results that NO cannot be generated by combining H₂-infused water with an acid and a source of nitrate without the base metal powder.

### Example 4

Compositions were prepared with two types of base metal powder:zinc metal powder and magnesium metal powder. Citric acid was used as the acid, and beet juice powder was used as a source of nitrate. Glucomannan and chitosan oligosaccharide were also added to form a hydrogel. These compositions were mixed with distilled water. Several other compositions were prepared without the base metal powder. Some of these other compositions were prepared with H₂-infused water or water having dissolved salts of zinc or magnesium to test whether the dissolved zinc and magnesium salts would affect the generation of NO. The ingredients of the compositions are shown in Table 3A. In addition to these ingredients, each composition also included 1 wt % glucomannan and 0.5 wt % chitosan oligosaccharide. The concentrations of NO, H₂, NO₂, NO₃, and the pH were measured and are shown in Table 3B.

TABLE 3A

| Test | Zn MP | Mg MP | Citric Acid | Beet Powder | |
|---|---|---|---|---|---|
| No. | (wt %) | (wt %) | (wt %) | (wt %) | Water/other |
| 1 | 0.15 | 0 | 0.30 | 1.00 | Distilled |
| 2 | 0.15 | 0 | 0.15 | 2.00 | Distilled |
| 3 | 0.15 | 0 | 0.15 | 1.00 | Distilled |
| 4 | 0 | 0.100 | 0.15 | 1.00 | Distilled |
| 5 | 0.50 | 0 | 0.15 | 1.00 | Distilled |
| 6 | 0.75 | 0 | 0.15 | 1.00 | Distilled |
| 7 | 0 | 0 | 0.15 | 1.00 | 892 ppb H₂ from |
| | | | | | H2MAX tablet |
| 8 | 0 | 0 | 0.15 | 1.00 | 900 ppb H₂ from |
| | | | | | electrolysis |
| 9 | 0 | 0 | 0.15 | 1.00 | Distilled |
| 10 | 0 | 0 | 0.15 | 1.00 | 991 ppb H₂ from |
| | | | | | HFactor |
| 11 | 0 | 0 | 0.15 | 1.00 | 928 ppb H₂ from |
| | | | | | HFactor + 0.2 wt % |
| | | | | | MgCl₂ |
| 12 | 0 | 0 | 0.15 | 1.00 | 946 ppb H₂ from |
| | | | | | HFactor + 0.2 wt % |
| | | | | | ZnSO₄ |
| 13 | 0 | 0 | 0.15 | 1.00 | 223 ppb H₂ from |
| | | | | | Susosa mineral water |

TABLE 3B

| Test | NO | H₂ | | NO₂ | NO₃ |
|---|---|---|---|---|---|
| No. | (ppb) | (ppb) | pH | (micromolar) | (ppm) |
| 1 | 2000+ | 504 | 3.96 | 870 | 360 |
| 2 | 1247 | 400 | 4.36 | 870 | 880 |
| 3 | 637 | 46 | 4.88 | 870 | 330 |
| 4 | 179 | 938 | 5.40 | 435 | 480 |
| 5 | 168 | 481 | 5.60 | 870 | 350 |
| 6 | 158 | 606 | 5.93 | 870 | 350 |
| 7 | 54 | 670 | 4.23 | 20 | 490 |
| 8 | 0 | 0 | 4.07 | 20 | 410 |
| 9 | 0 | 0 | 3.82 | 20 | 530 |
| 10 | 0 | 0 | 3.80 | 20 | 540 |
| 11 | 0 | 0 | 3.70 | 20 | 510 |
| 12 | 0 | 0 | 3.46 | 20 | 550 |
| 13 | 0 | 0 | 3.95 | 20 | 550 |

These results show the generation of NO and H₂ when the source of the nitrate is a natural product—beet root powder. Powder formulations were prepared gravimetrically and packaged in single dose packets. For testing, single dose packets were mixed with 100 mL of water. The water was either distilled water or water containing additional ingredients as shown in Table 3A. Measurements of NO, H₂, pH, NO₂, and NO₃ were then taken. The results indicate that lowering the pH by raising citric acid appears to be an effective means 10 of increasing NO generation in ZnMP—beet powder systems (test no. 1 compared to nos. 2-3). Higher levels of ZnMP (as in test nos. 5 & 6) do not lead to higher levels of NO or H₂, possibly because they lead to a higher pH, which ‘shuts down’ the reaction. The H₂MAX tablet water, which contains trace MgMP, is the only H₂-infused water that generates trace NO (54 ppb) and retains H₂-after mixing (i.e., continues generating H₂). This confirms that the presence of base metal can lead to sustaining H₂ generation. None of the other H₂-infused waters, with or without salts of Mg & Zn, can generate NO and sustain H₂ in these beet root powder gels (test nos. 8-13).

### Example 5

Although the Zn and Mg base metal powders studied here are both able to generate NO and H₂ in the presence of an acid and a source of nitrate, their time course of generating and sustaining H₂ and NO may differ. Therefore, a dose-response and time course study was performed on MgMP with sodium nitrate and citric acid in glucomannan/xanthan gum gels. Three compositions was prepared. The concentrations of MgMP in the compositions were 0.2 wt %, 0.4 wt %, and 0.8 wt %. All of the compositions also included 0.7 wt % citric acid, 0.1 wt % sodium nitrate, 7 wt % erithrytol, 0.7 wt % glucomannan, and 0.75 wt % xanthan gum mixed with distilled water. The concentrations of NO, H₂, NO₂, NO₃, and the pH were measured at different times after mixing the ingredients. The results are shown in Table 4.

TABLE 4

| MgMP | Time | | | | NO₂ | NO₃ |
|---|---|---|---|---|---|---|
| (wt %) | (hrs) | NO (ppb) | H₂ (ppb) | pH | (μM) | (ppm) |
| 0.2 | 0.1 | 1010 | 862 | 3.71 | 870 | 550 |
| 0.2 | 3 | 5 | 1418 | 9.6 | 870 | 460 |
| 0.4 | 0.1 | 830 | 909 | 4.2 | 870 | 430 |
| 0.4 | 3 | 176 | 1482 | 10.89 | 870 | 320 |
| 0.4 | 14 | 26 | 969 | 11 | 870 | 230 |
| 0.8 | 0.1 | 303 | 990 | 4.94 | 870 | 350 |
| 0.8 | 2 | 168 | 1407 | 11 | 870 | 270 |
| 0.8 | 13 | 151 | 1498 | 10.59 | 870 | 150 |
| 0.8 | 25 | 0 | 885 | 10.68 | 870 | 110 |

The dose-response at 0.1 hour indicates that NO generation decreases, as does NO₃, as MgMP increases. On the other hand, there are modest increases in H₂ at this time point. Since we have only qualitative estimates of NO₂ concentration, it cannot be determined. However, relatively elevated levels of NO₂ (greater than 870 μM) are being generated and sustained under these conditions. As the concentration of MgMP changes from 0.2 to 0.4 to 0.8%, the pH changes from 3.7 to 4.2 to 4.9. Thus, additional acid may be used to keep the pH low enough for optimal generation of NO in these complex systems. The time course aspect of these studies tells another interesting pH ‘story’. MgMP, in reacting with water, generates a high pH, which is associated with lower generation of NO and lower levels of NO₃ as well. Since the H₂ levels measured are pH-dependent, the effect of pH on the H₂ levels cannot be determined. It is possible that the increase in pH drives the conversion of NO₃ to form NO₂ (i.e., H₂+NO₃=NO₂+H₂O). If so, the reaction can be inhibited from proceeding to: H₂+NO₂=NO+H₂O since NO decreases as MgMP increases.

### Example 6

Given the interesting results presented in Example 5 for dose-response and time course of the parameters of interest due to MgMP, a more detailed study was conducted using ZnMP. A series of compositions was prepared with zinc metal powder, citric acid, and sodium nitrate in different amounts. Each composition also included 7 wt % erythritol, 0.7 wt % glucomannan, 0.75 wt % xanthan gum, and distilled water. The concentrations of NO, H₂, NO₂, NO₃, and the pH were measured at different time intervals. The ingredients of each composition are shown in Table 5A and the measurement results are shown in Table 5B.

TABLE 5A

| Test No. | ZnMP (wt %) | CA (wt %) | NaNO₃ (wt %) |
|---|---|---|---|
| 1 | 0.1 | 0.7 | 0.1 |
| 2 | 0.1 | 0.7 | 0.1 |
| 3 | 0.1 | 0.7 | 0.1 |
| 4 | 0.1 | 0.7 | 0.1 |
| 5 | 0.1 | 0.7 | 0.1 |
| 6 | 0.1 | 0.7 | 0.1 |
| 7 | 0.1 | 0.7 | 0.1 |
| 8 | 0.4 | 0.7 | 0.1 |
| 9 | 0.4 | 0.7 | 0.1 |
| 10 | 0.4 | 0.7 | 0.1 |
| 11 | 0.8 | 0.7 | 0.1 |
| 12 | 0.8 | 0.7 | 0.1 |
| 13 | 0.8 | 0.7 | 0.1 |
| 14 | 0.19 | 0 | 0.1 |
| 15 | 0.19 | 0 | 0.1 |
| 16 | 0.19 | 0.18 | 0.1 |
| 17 | 0.19 | 0.18 | 0.1 |
| 18 | 0.19 | 0.7 | 0.05 |
| 19 | 0.19 | 0.7 | 0.05 |
| 20 | 0.19 | 0.7 | 0.05 |
| 21 | 0.19 | 0.7 | 0.20 |
| 22 | 0.19 | 0.7 | 0.20 |
| 23 | 0.19 | 0.7 | 0.20 |
| 24 | 0.19 | 0.7 | 0.30 |
| 25 | 0.19 | 0.7 | 0.30 |
| 26 | 0.19 | 0.7 | 0.30 |

TABLE 5B

| Test | Time | | | | NO₂ | NO₃ |
|---|---|---|---|---|---|---|
| No. | (hrs) | NO (ppb) | H₂ (ppb) | pH | (μM) | (ppm) |
| 1 | 0.1 | 1783 | 248 | 3.08 | 870 | 480 |
| 2 | 1 | 1771 | 0 | 3.28 | 870 | 460 |
| 3 | 2 | 1633 | 0 | 3.24 | 870 | 430 |
| 4 | 7 | 1030 | ND | ND | ND | 380 |
| 5 | 17 | 771 | ND | ND | ND | 400 |
| 6 | 22 | 660 | ND | ND | ND | 350 |
| 7 | 30 | 152 | ND | ND | ND | 400 |
| 8 | 0.1 | 1065 | 625 | 4.0 | 870 | 110 |
| 9 | 4 | 0 | 795 | 6.49 | 220 | 78 |
| 10 | 11 | 0 | 1000 | 6.6 | 20 | 88 |
| 11 | 0.1 | 0 | 1330 | 5.15 | 870 | 81 |
| 12 | 5 | 0 | 1521 | 7.56 | 20 | 110 |
| 13 | 12 | 0 | 983 | 6.84 | 20 | 100 |
| 14 | 0.1 | 0 | 728 | 5.62 | 870 | 600 |
| 15 | 7 | 0 | 772 | 5.52 | 220 | 580 |
| 16 | 0.1 | 125 | 663 | 4.14 | 870 | 390 |
| 17 | 7 | 0 | 260 | 7.22 | 870 | 230 |
| 18 | 0.1 | 152 | 375 | 3.36 | 220 | 81 |
| 19 | 5 | 0 | 401 | 3.69 | 20 | 86 |
| 20 | 17 | 0 | 0 | 3.71 | 20 | 69 |
| 21 | 0.1 | 1626 | 287 | 3.36 | 870 | 660 |
| 22 | 5 | 1175 | 0 | 3.77 | 870 | 580 |
| 23 | 17 | 952 | 0 | 3.69 | 870 | 530 |
| 24 | 0.1 | 1733 | 631 | 3.36 | 870 | 1200 |
| 25 | 5 | 1034 | 0 | 3.67 | 870 | 990 |
| 26 | 17 | 890 | 0 | 3.65 | 870 | 650 |

The results for varying the concentration of ZnMP, at constant CA and NaNO₃, when evaluated at the earliest point of the Study, indicate a drop in the level of NO as ZnMP is increased (test nos. 1, 8 and 11). This trend is similar to that found when evaluating the MgMP formulations of Example 5. A similar downward trend is seen in the level of NO₃. In contrast, there is an increase in pH—but not as dramatic as seen for the upward trend driven by increasing MgMP.

Observing the time course of parameters associated with the 0.1% ZnMP, 0.7% CA, 0.1% NaNO₃ formulation (test nos. 1-7), it can be seen that NO and NO₃ progressively, but slowly decrease with time, while H₂ is dissipated within one hour. NO₂ remains at 870+μM level. It is possible that H₂ is being consumed by the generation of NO and NO₂. Observing the time course of parameters associated with the 0.4% ZnMP, 0.7% CA, 0.1% NaNO₃ formulation (test nos. 8-10), it can be seen that NO rapidly decreases while NO₃ is initially dissipated to a low level (from a potential high of around 500 ppm). In contrast, H₂ increases over this time period. Surprisingly, NO₂ decreases from 870 to 220 to 20 as time proceeds. The cause of the disappearance of NO, NO₂ and NO₃ may lie in the increase in the pH with time. That is, at the higher pH, complexes with zinc or components of the gel could take place masking the nitrogen components from detection.

Observing the time course of parameters associated with the 0.8% ZnMP, 0.7% CA, 0.1% NaNO₃ formulation (test nos. 11-13), it can be seen that NO is not generated at any time period. H₂ slowly dissipates. NO₂ decreases from 870 μM to zero as time proceeds. Again, the decrease in NO₂ may be due to increasing pH. Thus, ZnMP, and other base metals, can have a biphasic response. At low dose, in defined acidic pH conditions, with nitrate, the base metal powder contributes to the generation of NO while at defined higher levels and higher pH it inhibits the generation of NO.

The formulations of test no. 14-17 show the effect of raising citric acid (CA) in the 0.19% ZnMP, 0.1% NaNO₃ formulation from 0.0 to 0.18%. The results demonstrate that the pH decreases from 5.6 to 4.1. This results in a modest increase in NO generation from zero to 125 ppb; a slight decrease in H₂ generation; and a large decrease in NO₃. Over time, in the presence of 0.18% CA, the pH rises to 7.2 (test no. 17)—a pH where generation of NO or H₂ is not expected. These results suggest that NO and H₂ will not be generated when a pH greater than 4.1 prevails.

Test nos. 18-26 present the results of studies varying sodium nitrate (NO₃). At the initial measurement (i.e., 0.1 hour), increasing the concentration of NO₃ from 0.05 to 0.20 to 0.30% markedly raises NO from 152 to 1626 to 1733 ppb. A modest amount of H₂ is generated. The pH stabilizes at 3.4. The amount of NO₃ generated also increases according to the administered dose. These results suggest that increasing NO₃ can increase NO—not at the expense of H₂.

Over time, NO is remarkably persistent while H₂ is rapidly diminished. NO₃ is diminished to a lesser extent. The changes in the NO₃ concentration do not have an appreciable effect on changing the pH.

### Example 7

It has been proposed, above, that ZnMP, as well as other base metals (e.g., MgMP) catalyzes the generation of NO by a two—step reaction process:H₂+NO₃=H₂O+NO₂ and H₂+NO₂=NO+H₂O. An experiment was performed to study the factors involved in conversion of NO₂ to NO. A series of compositions was prepared with various amounts of ascorbic acid, citric acid, sodium nitrite, and zinc metal powder. All of the formulations also included 7 wt % erythritol, 0.7 wt % glucomannan, 0.7 wt % xanthan gum. Table 6A shows the ingredients of the compositions. Table 6B shows the measured concentrations of NO, H₂, NO₂, and the pH.

TABLE 6A

| Test no. | AA (wt %) | CA (wt %) | NaNO₂ (wt %) | ZnMP |
|---|---|---|---|---|
| 1 | 1.0 | 1.0 | 0.1 | 0.25 |
| 2 | 1.0 | 0 | 0.1 | 0.25 |
| 3 | 1.0 | 0 | 0.1 | 0 |
| 4 | 1.0 | 2.0 | 0.1 | 0.25 |
| 5 | 0.0 | 2.0 | 0.1 | 0.25 |
| 6 | 1.0 | 0 | 0.1 | 0.25 |

TABLE 6B

| Test No. | NO (ppb) | H₂ (ppb) | pH | NO₂ (μM) |
|---|---|---|---|---|
| 1 | 2000 | 400 | 3.9 | 870 |
| 2 | 0 | 274 | 7.13 | 870 |
| 3 | 387 | 0 | 6.12 | 870 |
| 4 | 2000 | 1185 | 3.35 | 20 |
| 5 | 2000 | 488 | 2.85 | 870 |
| 6 | 0 | NA | 7.35 | NA |

Test no. 3 confirms that ascorbic acid plus nitrite (NO₂) can produce NO. Comparing this test to that of no. 5, it can be observed that the reaction of 2% citric acid with 0.1% nitrite in the presence of ZnMP allows the production of much more NO, as well as H₂. The contribution of low pH can be seen here by comparing test no. 4 with test no. 6 where the pH rises to 7.35. Under this condition, no NO is generated.

Overall, the results demonstrate that at a pH below 4.0, ZnMP can catalyze the conversion of nitrite to NO, whether or not ascorbic acid is present (see tests no. 1, 4, and 5).

### Example 8

The above examples point to the feasibility of generating both nitric oxide (NO) and molecular hydrogen (H₂) in nitrite and/or nitrate formulations, some also containing activators of NO and H₂. That is, a more acidic pH can contribute to the H₂ and NO generative reactions. Table 7 displays additional experiments that were performed to show effective nitrite (#1-8) and nitrate (#9-17) formulations can readily generate NO and discernable erythema (skin redness)—a marker for vasodilation and increased blood flow. The nitrate formulations readily generate NO—but with less or no sign of erythema. However, some formulations containing high doses of nitrate and activators demonstrate discernable erythema.

Results reported previously demonstrated that a gel formula containing 0.25% ZnMP, 0.7% citric acid and 0.1% sodium nitrate can generate nitric oxide (NO)—both in vitro and on skin. However, application of this formulation to the skin surface failed to generate significant erythema (Score ‘1’ or higher—for significance). Erythema can indicate vasodilation and increased blood flow. This perceived lack of efficacy may have been due to the nature of the formulation.

Briefly, in vitro studies were carried out by mixing a single dose packet of the test powder with 100 mL of distilled water until a gel starts to form, i.e., about 30-seconds. After about 10-minutes, the NO emanating from the plastic cup was measured with the Interscan GasD 800 NO detector. Also, for some formulations test strips (Human N or Berkeley) were used to test for nitrite (not shown here), the Trustlex for H₂ and the Extec II for pH.

For in vivo skin studies, a 1-gram sample was smoothed onto a 15 cm diameter site on the inner aspect of the right forearm and allowed to be present for 5—minutes before taking the same measurements as done for the in vitro studies. 5 The results are summarized in Tables 7A-7B.

TABLE 7A
Nitrite Compositions

| | Formula No. | | | | | | | |
|---|---|---|---|---|---|---|---|---|
| | 1 | 2 | 3 | 4 | 5 | 6 | 7 | 8 |
| | Grams | Grams | Grams | Grams | Grams | Grams | Grams | Grams |
| Ingredient | | | | | | | | |
| ascorbic acid | 1.0 | 1.0 | 1.0 | 1.0 | 1.0 | 1.0 | 1.0 | 1.0 |
| Citric acid | 1.0 | 0.5 | 0.5 | 0.5 | 0.5 | 0.5 | 0.5 | 1.0 |
| Zinc MP | 0.25 | 0.19 | | 0.19 | 0.19 | 0.19 | 0.19 | 0.19 |
| Na Nitrite | 0.10 | 0.1 | 0.1 | 0.02 | 0.05 | 0.07 | 0.09 | 0.10 |
| Results | | | | | | | | |
| pH solution | 3.90 | 4.33 | 4.24 | 4.14 | 4.21 | 4.24 | 4.39 | 3.86 |
| pH put on skin | 4.06 | 4.72 | 4.42 | 4.25 | 4.42 | 4.54 | 4.66 | 4.06 |
| In Vitro H2, ppb | 400 | 425 | 0 | 864 | 589 | 624 | 520 | 646 |
| Skin H2, ppb | 20 | 789 | 0 | 568 | 760 | 521 | 492 | 493 |
| ppb In vitro NO | 2000+ | 2000+ | 2000+ | 620 | 2000+ | 2000+ | 2000+ | 2000+ |
| ppb NO on skin | 369 | NotDone | 582 | 0 | 1375 | 980 | 2000+ | 1214 |
| Max Erythema | 2+ | 3 | 2+ | 0+ | 3 | 2+ | 3 | 3+ |
| Min. Ery. Lasts | 30 | 60 | 20 | 0 | 25 | 22 | 65 | 180 |

TABLE 7B
Nitrate Compositions

| | Formula No. | | | | | | | | |
|---|---|---|---|---|---|---|---|---|---|
| | 9 | 10 | 11 | 12 | 13 | 14 | 15 | 16 | 17 |
| | Grams | Grams | Grams | Grams | Grams | Grams | Grams | Grams | Grams |
| Ingredient | | | | | | | | | |
| ascorbic acid | 1.0 | 1.0 | 1.0 | | | | | | |
| an. Citric acid | | | | 2.0 | 2.0 | 2.0 | 0.70 | 0.70 | 0.70 |
| Zinc MP | | 0.25 | 0.19 | 0.76 | 0.76 | 0.76 | | 0.19 | 0.76 |
| Mg MP | | | | | | | 0.2 | | |
| Na Nitrate | 0.1 | 0.1 | 0.1 | 0.30 | 0.20 | | | | |
| Mg Nitrate | | | | | | 0.4 | 0.1 | 0.1 | 0.1 |
| Results | | | | | | | | | |
| pH solution | 6.03 | 6.88 | 6.20 | 3.34 | 3.36 | 3.64 | 3.54 | 3.21 | 3.15 |
| pH put on skin | | 6.08 | 6.24 | 3.85 | 3.77 | 3.88 | 4.03 | 3.58 | 3.50 |
| In Vitro H2, ppb | 0.0 | 410 | 796 | 846 | 1053 | 792 | 854 | 547 | 369 |
| Skin H2, ppb | 0.0 | 660 | 607 | | 717 | 800 | 795 | 190 | 267 |
| ppb In vitro NO | 0.0 | 0 | 0 | 2000+ | 987 | 2000+ | 645 | 2000+ | 1198 |
| ppb NO on skin | 0.0 | 18 | 0 | 1093 | 98 | 563 | 78 | 255 | 167 |
| Max Erythema | 0 | 0 | 0 | 2 | 0+ | 2+ | 0+ | 0+ | 1 |
| Min. Ery. Lasts | 0 | 0 | 0 | 48 | 0 | 60 | 0 | 0 | 60 |

Formulas #1-8 contain different concentrations of sodium nitrite. Each formula 1-17 also contains 7 g. erythritol, 0.70 g. konjac glucomannan, 0.70 g. xanthan gum. All of these formulas except 4 produce discernable erythema on skin (0+ erythema (Ery) indicates the perceived erythema is uncertain). Of interest is #5.

Only nitrate formulas #12, 14 and 17 generate definitive erythema, although formulas 13, 15 and 16 may have an uncertain effect of producing erythema. Formula 14, containing the highest doses of ZnMP, citric acid & Mg(NO₃)₂ produce the most erythema. At a high dose of the ‘active’ ingredients, non-irritating erythema is produced. It is now established that a combination of a nitrate, a base metal powder and an organic acid can produce erythema which can be attributed to NO. Irritation as a cause of the perceived erythema is unlikely—since this erythema rapidly diminishes—and does not leave any longer-term signs irritation—such as dry, flaky skin.

These studies demonstrate that: a) nitrite, in the presence of ascorbic acid, with or without (See Formula #3) a base metal powder—generates NO and erythema; b) nitrite with ZnMP appears to generate stronger erythema; c) nitrate when formulated with citric acid (but not with ascorbic acid) and a base metal, at an acidic pH generates skin surface NO as well as in vitro NO. Although, in vivo, the combination of a nitrate, citric acid and a base metal powder generates an amount of NO—on parity with that generated by nitrite in the presence of acidic ascorbic acid, its effect on generating erythema appears to be much weaker.

To attempt to understand the relative ‘weak’ effect of the nitrate-citric acid-base metal combination on production of erythema it is possible that nitric oxide itself does not readily penetrate skin. Nitrite may penetrate skin and react with ascorbic acid, in skin, producing NO in the lower layers of skin—thereby affecting vasodilation & resultant erythema. In acidic H₂-nitrate formulations, nitrate may be rapidly converted to NO—such that nitrite is not bioavailable for skin penetration. Thus, NO may be detected only on the surface of the skin. Further, at the highest doses of nitrate and activators, some nitrite may survive to penetrate to the lower layers of the skin, thus available, there, for conversion to NO.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A composition comprising a base metal powder, a nitrogen-containing salt, and an acid.

2. The composition of claim 1, wherein the composition, when mixed with water, generates and sustains at least one of nitric oxide or molecular hydrogen.

3. The composition of claim 1, wherein the base metal powder comprises zinc, magnesium, calcium, or a combination thereof.

4. The composition of claim 1, wherein the base metal powder is present at a concentration from 0.005 wt % to 50 wt %.

5. The composition of claim 1, wherein the acid is present at a concentration from 0.05 wt % to 70 wt %.

6. The composition of claim 1, wherein the acid is an organic acid.

7. The composition of claim 6, wherein the acid is citric acid.

8. The composition of claim 1, wherein the nitrogen-containing salt is present at a concentration from 0.01 wt % to 50 wt %.

9. The composition of claim 1, wherein the nitrogen-containing salt is a nitrate salt, a nitrite salt, or a combination thereof.

10. The composition of claim 1, wherein the nitrogen-containing salt is a nitrate salt.

11. The composition of claim 1, wherein the nitrogen-containing salt comprises a cation selected from the group consisting of sodium, potassium, magnesium, calcium, zinc, ammonium, aluminum, barium, bismuth, copper, iron, lithium, manganese, silver, and combinations thereof.

12. The composition of claim 1, wherein the nitrogen-containing salt is present as a part of a natural product selected from the group consisting of a botanical powder, a vegetable powder, a fruit powder, and combinations thereof.

13. The composition of claim 12, wherein the natural product is a botanical powder selected from the group consisting of green coffee bean powder, danshen root powder (Radix saliva mitorrhizae), snake gourd fruit powder (Fructus trichosanthis), longstamen onion bulb powder (Bulbus allii macrostemi), sanchi powder (Radix notoginseng), ginseng powder (Radix ginseng), bing pian powder (Borneolum syntheticum), and borneol powder (Cinnamomum), and combinations thereof.

14. The composition of claim 12, wherein the natural product is a vegetable powder selected from the group consisting of arugula powder, beet powder, cabbage powder, celery powder, cress powder, fennel powder, leek powder, spinach powder, lettuce powder, mustard green powder, parsley powder, swiss chard powder, leafy chicory powder, kohlrabi powder, radish powder, and combinations thereof.

15. The composition of claim 12, wherein the natural product is a fruit powder selected from the group consisting of watermelon powder, apple powder, banana powder, grape powder, kiwi powder, nectarine powder, peach powder, pomegranate powder, pear powder, orange powder, and combinations thereof.

16. The composition of claim 12, wherein the natural product is present at a concentration from 0.5 wt % to 80 wt %.

17. The composition of claim 1, further comprising a hydrogel-forming polymer selected from the group consisting of konjac glucomannan, xanthan gum, chitosan, chitosan oligosaccharide, kappa carrageenan, lambda carrageenan, sulfated polysaccharide, alginate, pectin, and combinations thereof.

18. The composition of claim 17, wherein the hydrogel-forming polymer is present at a concentration from 5 wt % to 70 wt %.

19. A method of treating a condition in a subject comprising administering to the subject the composition of claim 1.

20. The method of claim 19, wherein the composition is administered topically.

21. The method of claim 19, wherein the composition further comprises water and wherein the composition is in a form of a hydrogel, paste, gum, or liquid.

22. The method of claim 19, wherein the condition is aging skin, diabetic ulcers, burns, bacterial infection, viral infection, fungal infection, hair loss, mouth ulcers, herpes infection, gingivitis, periodontal disease, halitosis, esophagitis, lower esophageal spasms, esophageal ulcers, gastroparesis, gastric ulcers, SIBO (small intestinal bacterial overgrowth), Helicobacter pylori infection, or a combination thereof.

23. The method of claim 19, wherein the composition generates NO at a concentration from 50 ppb to 150 ppm, in parts by weight with respect to a total weight of the composition, for a time period from 0.1 hour to 50 hours.

24. The method of claim 19, wherein the composition generates H₂ at a concentration from 50 ppb to 3,000 ppb, in parts per billion by weight with respect to a total weight of the composition, for a time period from 0.1 hour to 50 hours.
